(19) United States
(12) Patent Application Publication
(10) Pub. No.: US 2012/0288928 A1
(43) Pub. Date: Nov. 15, 2012

(54) PHOTOBIOREACTOR FOR CULTURING MICROALGAE USING HOLLOW FIBER MEMBRANE

(75) Inventors: Bum Suk Jung, Yongin (KR); Joo Hwan Lim, Incheon (KR); Dae Young Goh, Busan (KR); Soo Hyun Ha, Seoul (KR); Won Bae Lee, Seoul (KR); Shin Tae Bae, Anyang (KR)

(73) Assignees: Myongji University Industry and Academia Cooperation, Yongin (KR); Hyundai Motor Company, Seoul (KR)

(21) Appl. No.: 13/212,406

(22) Filed: Aug. 18, 2011

(30) Foreign Application Priority Data
May 12, 2011 (KR) 10-2011-0044309

(51) Int. Cl. C12M 1/04

(52) U.S. Cl. 435/292.1

(57) ABSTRACT

Disclosed is a high-speed photobioreactor for culturing microalgae using a hollow fiber membrane. More particularly a high-speed photobioreactor for culturing microalgae using a hollow fiber membrane capable of facilitating growth of microalgae and maximizing carbon dioxide fixation by increasing the rate of carbon dioxide saturation in a culture medium. Specifically, a high-speed photobioreactor for culturing microalgae using a hollow fiber membrane includes a reactor main body for culturing microalgae; a hollow fiber membrane module for supplying carbon dioxide into a culture medium in the reactor main body; a culture medium circulation pump for circulating the culture medium; and a defoamer for removing foams produced in the culture medium.

### CROSS-REFERENCE TO RELATED APPLICATIONS

This application claims priority under 35 U.S.C. §119 to Korean Patent Application No. 10-2011-0044309, filed on May 12, 2011, in the Korean Intellectual Property Office, the disclosure of which is incorporated herein by reference in its entirety.

### BACKGROUND

(a) Technical Field

The present invention relates to a high-speed photobioreactor for culturing microalgae using a hollow fiber membrane. More particularly, the present invention relates to a high-speed photobioreactor for culturing microalgae using a hollow fiber membrane capable of facilitating growth of microalgae and maximizing carbon dioxide fixation by increasing the rate of carbon dioxide saturation in a culture medium.

(b) Background Art

Various attempts have been made to solve the planet wide environmental problems associated with global warming and fossil fuel depletion. Among some of these attempts is a method of biologically reducing CO₂ and producing biodiesel by utilizing photosynthesis of microalgae which has proven advantageous in that it is attainable at normal temperature and pressure and is based on the carbon cycle of nature. Thus, it is considered as the most practical solution for greenhouse gas reduction.

For a technology based on the photosynthesis of microalgae to be a successful solution, a microalgal species having an excellent CO₂ absorbing ability should be selected and a photobioreactor for culturing it must be developed. In general, the conventional apparatuses for culturing microalgae can be classified into an open pond system and a closed system. Since the open pond system uses an open trench or pond, the initial investment cost is fairly low. However, a large installation space is required because the productivity per unit area is also low and it is difficult to control the amount of nutrients, temperature, pH and other factors that are necessary for growth of microalgae.

To overcome the problems associated the open pond system, a closed systems is sometime used to allow growth of microalgae in high densities in a small-sized reactor so that it can be actively studied. Typically, these existing apparatuses for culturing microalgae consist of a nutrient supplier, a microalgal photobioreactor, and a harvester. The nutrient supplier supplies nutrients and water necessary for the growth of microalgae, and the microalgal photobioreactor allows the microalgae to photosynthesize using natural natural/artificial light so as to fix CO₂. The harvester, as its name suggests, removes the grown microalgae.

Among these constituents, the microalgal photobioreactor, where the fixation of CO₂ is actually achieved, is the core element of the biological CO₂ fixation process. Usually, it takes 9-10 days for the microalgae to grow from the initial concentration to the final concentration. Microalgae grows so slowly because CO₂ gas is injected into the reactor simply by a bubbling method and thus does not ensure a lengthy contact time of CO₂ with the microalgae due to the low solubility of CO₂ in water. Hence, there is a low residence time in the culture medium. Additionally, since the gas emitted from the culture medium is not completely CO₂-free, an additional collecting device is required to reuse the gas from the culture medium. Furthermore, there are also problems in reuse of the water and harvesting of the microalgae since the culture medium and the microalgae should be managed separately.

### SUMMARY

The present invention is directed to providing a high-speed photobioreactor for culturing microalgae using a hollow fiber membrane capable of facilitating the growth of microalgae and maximizing carbon dioxide by using a hollow fiber membrane having a large membrane surface area and thus increases the saturation rate of carbon dioxide in the culture medium.

In one general aspect, the present invention provides a high-speed photobioreactor for culturing microalgae using a hollow fiber membrane which includes a reactor main body for culturing microalgae; a hollow fiber membrane module for supplying carbon dioxide into a culture medium in the reactor main body; a culture medium circulation pump for circulating the culture medium; and a defoamer for removing foams produced in the culture medium.

The reactor main body may be equipped with a separation membrane which separates a microalgae-mixed culture medium mixed with microalgae and a circulating culture medium which includes carbon dioxide supplied from the hollow fiber membrane module and transfers the carbon dioxide included in the circulating culture medium to the microalgae-mixed culture medium by a concentration gradient.

A light source provided outside the reactor main body may be configured to illuminate light having a wavelength that activates photosynthesis into the reactor main body. Further, one or more stirrers may be provided in the reactor main body to ensure flowability of the microalgae.

More specifically, the separation membrane may have pores of a size of about 0.4 nm or smaller to block the movement of the microalgae. A hollow fiber membrane of the hollow fiber membrane module may be a hydrophobic membrane having pores of a size of about 0.1 nm or smaller. The hollow fiber membrane of the hollow fiber membrane module may also be a membrane with a porosity of about 10-40%.

Furthermore, another hollow fiber membrane module may be provided between the hollow fiber membrane module and the reactor main body, and a gas inlet of the another hollow fiber membrane module may be connected to a gas outlet of the hollow fiber membrane module to increase contact time of carbon dioxide with the culture medium.

Since the high-speed photobioreactor using a hollow fiber membrane according to the present invention is capable of supplying CO₂ necessary for the growth of microalgae at high speed to the culture medium and of separating the microalgae-mixed culture medium from the microalgae-free culture medium using the separation membrane, it is easy to supply nutrients and remove harmful substances, thus facilitating the growth of the microalgae. Furthermore, scaling up is possible through modularization and microalgal growth and carbon dioxide fixation can be maximized.

The above and other aspects and features of the present invention will be described infra.

### BRIEF DESCRIPTION OF THE DRAWINGS

The above and other objects, features and advantages of the present invention will now be described in detail with reference to certain exemplary embodiments thereof illustrated in the accompanying drawings which are given hereinbelow by way of illustration only, and thus are not limitative of the invention, and wherein:

FIG. 1 shows a configuration of a high-speed photobioreactor for culturing microalgae using a hollow fiber membrane according to an exemplary embodiment of the present invention;

FIG. 2 shows a configuration of a high-speed photobioreactor for culturing microalgae using a hollow fiber membrane according to another exemplary embodiment of the present invention;

FIGS. 3a and 3b shows configuration of high-speed photobioreactors modularized according to the exemplary embodiment of the present invention; and

FIG. 4 shows a result of, after supplying carbon dioxide at a constant flow rate to a culture medium using a hollow fiber membrane module according to the exemplary embodiment of the present invention or using an existing bubbling reactor, comparing the concentration of carbon dioxide dissolved in each culture medium.

It should be understood that the appended drawings are not necessarily to scale, presenting a somewhat simplified representation of various preferred features illustrative of the basic principles of the invention. The specific design features of the invention as disclosed herein, including, for example, specific dimensions, orientations, locations and shapes, will be determined in part by the particular intended application and use environment.

In the figures, reference numerals refer to the same or equivalent parts of the disclosure throughout the several figures of the drawings.

### DETAILED DESCRIPTION

Hereinafter, reference will now be made in detail to various embodiments of the present invention, examples of which are illustrated in the accompanying drawings and described below. While the invention will be described in conjunction with exemplary embodiments, it will be understood that the present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The present invention relates to a high-speed photobioreactor for culturing microalgae using a hollow fiber membrane. By increasing the saturation rate of carbon dioxide supplied to a culture medium by using a hollow fiber membrane having an increased membrane surface area, growth of microalgae can be facilitated and carbon dioxide fixation can be increased.

In addition to the increase of the saturation rate of carbon dioxide in the culture medium, use of the hollow fiber membrane allows removal of oxygen produced during the culturing of the microalgae, thereby facilitating the metabolism of the microalgae. Further, by providing a separation membrane capable of blocking movement of the microalgae in the reactor main body, the transport of the culture medium can be controlled independently and the efficiency of the entire system can be improved.

That is to say, the present invention allows for faster supply of carbon dioxide gas using a hollow fiber membrane than the conventional bubbling employed in the existing art. Furthermore, by supplying carbon dioxide to the microalgae as a nutrient for photosynthesis using a light source (natural or artificial light) through the separation membrane provided in the reactor main body, the concentration of carbon dioxide dissolved in the culture medium mixed with the microalgae (hereinafter, referred to as microalgae-mixed culture medium) can be controlled and thus prevents the microalgae from coming out of the reactor main body and therefore prevents attachment of the microalgae to the hollow fiber membrane module. Further, by circulating the culture medium below the separation membrane between the reactor main body and the hollow fiber membrane module so that the concentration of carbon dioxide is maintained as a constant and separating the culture medium (hereinafter, referred to as circulating culture medium) from the microalgae-mixed culture medium, the microalgae and the culture medium may be managed separately.

Since both the reactor main body and the hollow fiber membrane module can be modularized, the high-speed photobioreactor of the present invention can be scaled up easily and thus carbon dioxide fixation can be maximized.

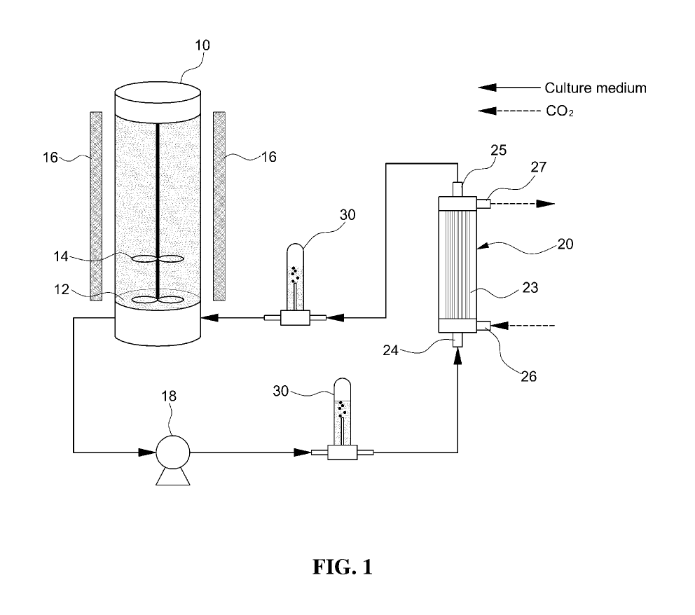

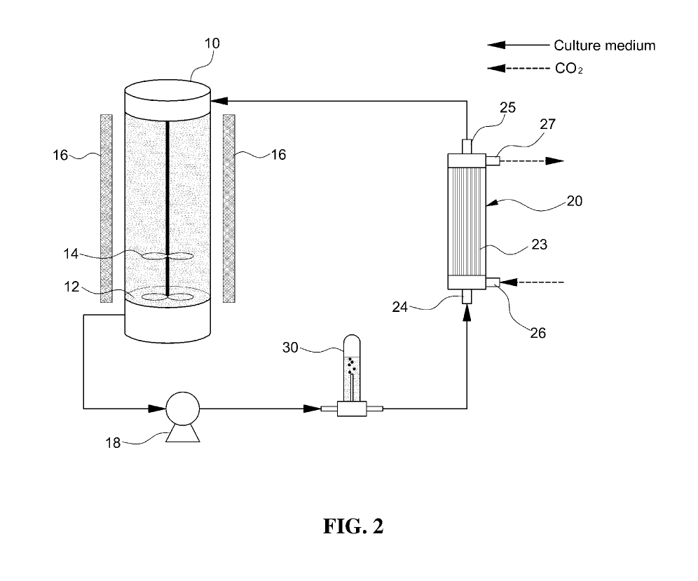

As shown in FIG. 1 and FIG. 2, a high-speed photobioreactor according to an illustrative embodiment of the present invention includes a reactor main body 10 having a cylindrical shape with a predetermined volume; a hollow fiber membrane module 20 for transport of material, such as supply of carbon dioxide to and removal of oxygen from a culture medium; a defoamer 30 for preventing foaming of a culture medium supplied to the reactor main body 10 and the hollow fiber membrane module 20; a light source 16 provided outside the reactor main body 10 and illuminating light with a wavelength suitable for culturing of plants into the reactor main body 10; and a culture medium circulation pump 18 for circulating the culture medium.

The reactor main body 10 is designed to culture microalgae therein and is filled with the culture medium for supplying nutrients. At the bottom of the reactor main body 10, a plate-type separation membrane 12 capable of separating a microalgae-mixed culture medium from a circulating culture medium and blocking movement of the microalgae is provided.

The separation membrane 12 can be a plate-type membrane with pores of a size of about 0.4 nm or smaller so that the microalgae cannot pass therethrough while at the same time allowing the culture medium to pass through the separation membrane 12. That is to say, the separation membrane 12 allows transport of material (e.g., carbon dioxide and oxygen) while separating the microalgae from the culture medium (especially, the circulating culture medium) in the reactor main body 10. The separation membrane 12 preferably has a diameter corresponding to the inner diameter of the reactor main body 10.

The provision of the separation membrane 12 in the reactor main body 10 allows the supply of carbon dioxide necessary for photosynthesis of the microalgae from the circulating culture medium at the bottom portion of the reactor main body 10 to the microalgae-mixed culture medium thereabove as well as the transport of oxygen resulting from the photosynthesis to the circulating culture medium so that the oxygen can be removed. That is to say, the separation membrane 12 allows for separation of the microalgae-mixed culture medium from the carbon dioxide-containing circulating culture medium supplied from the hollow fiber membrane module 20 as well as transport of carbon dioxide from the circulating culture medium to the microalgae-mixed culture medium via a concentration gradient.

In other words, by a concentration gradient of the culture media separated by the separation membrane 12, the carbon dioxide supplied from the hollow fiber membrane module 20 is transported to the microalgae-mixed culture medium. The control of material transport of the culture medium by the concentration gradient improves the efficiency of the entire system. Accordingly, the separation membrane 12 may be a membrane capable of blocking the movement of the microalgae but also allowing the transport of various nutrients required by the microalgae as well as harmful substances such as carbon dioxide, oxygen, or the like.

Due to this separation by the separation membrane 12 in the reactor main body 10, fouling of a hollow fiber membrane 23 that may occur, for example, by the microalgae when carbon dioxide is supplied to the hollow fiber membrane module 20 may be prevented, the microalgae may be harvested conveniently, and it becomes easier to reuse the remaining culture medium and supplement insufficient nutrients then would be possible in the conventional closed systems.

Further, a stirrer 14 may be provided in the reactor main body 10 to prevent flocculation and fouling of the separation membrane 12 caused by the concentration gradient. The stirrer 14 may be provided above the separation membrane 12 in singular or plural numbers so as to ensure sufficient flowability of the microalgae by stirring the culture medium, especially the microalgae-mixed culture medium, in the reactor main body 10, thereby preventing flocculation and fouling of the separation membrane 12.

The light source 16 is a lamp provided close to the reactor main body 10 to illuminate light having a wavelength that activates photosynthesis suitable for culturing of plants. Specifically, the light source 16 may emit light having a wavelength of about 450 nm or about 660 nm, which activates chlorophylls for photosynthesis. The light source 16 supplies light energy from outside the reactor main body 10 together with natural light. The intensity of the light is about 200 μmol in m⁻²s⁻¹, which is suitable for photosynthesis.

The hollow fiber membrane module 20 includes a plurality of hollow fiber membranes 23 inserted in a tube-shaped module housing in parallel to the module housing. Both ends of the hollow fiber membrane 23 may be fixed to the module housing by an epoxy layer.

The hollow fiber membrane 23 is made of a hydrophobic material so that the pores of the membrane are not wet by the culture medium to ensure good material transport. Also, the hollow fiber membrane may have pores of a predetermined size and a porosity of about 10-40%. For example, the hollow fiber membrane 23 may be a hydrophobic membrane with pores of a size of about 0.1 nm or smaller.

The carbon dioxide-containing gas supplied to the hollow fiber membrane module 20 may be pure carbon dioxide or a mixture of carbon dioxide and nitrogen or carbon dioxide and air, depending on growth level and concentration of the microalgae.

Most of the existing closed photobioreactors use an aeration tube equipped at the reactor main body to supply carbon dioxide as bubbles. However, in this case, the gas emitted from the culture medium includes a considerable amount of carbon dioxide and it is difficult for the reactor to completely remove the supplied carbon dioxide. Also, the supply of carbon dioxide is often slow and the removal of the oxygen produced from photosynthesis by the microalgae is not considered.

On the other hand, the photobioreactor according to the present invention is capable of effectively supplying carbon dioxide to the culture medium due to the increased effective membrane surface area provided by the fine pores of the hollow fiber membrane 23 of a size of about 0.1 nm or smaller. Furthermore, since it can remove the oxygen produced from the photosynthesis by the microalgae, the metabolism by the microalgae can be facilitated.

At both ends of the hollow fiber membrane module 20, a culture medium inlet 24 for inflow of the circulating culture medium, a culture medium outlet 25 for discharge of the circulating culture medium, a gas inlet 26 for inflow of the carbon dioxide-containing gas and, a gas outlet 27 for discharge of the carbon dioxide-containing gas mixed with oxygen emitted from the culture medium are provided.

Through the culture medium inlet 24, the circulating culture medium to which the oxygen produced from the photosynthesis of the microalgae has been transferred after the carbon dioxide has been supplied to the culture medium in the reactor main body 10 is flown in. Through the culture medium outlet 25, the circulating culture medium which is saturated by the carbon dioxide supplied through the gas inlet 26 as it passes through the hollow fiber membrane 23 and from which oxygen has been discharged out of the hollow fiber membrane 23 is discharged.

That is to say, when the circulating culture medium wherein the level of carbon dioxide has decreased and that of oxygen has increased as a result of the photosynthesis by the microalgae is flown in, the hollow fiber membrane 23 serves to remove oxygen from the circulating culture medium and increase the concentration of the carbon dioxide.

The hollow fiber membrane 23 usually serves as a device for supplying carbon dioxide and gas, but, when the concentration of oxygen in the culture medium (circulating culture medium) increases as a result of the photosynthesis, it may serve as a module that removes the oxygen dissolved in the reactor main body 10 that has passed through the separation membrane 12 while nitrogen or the mixture gas is transported.

The defoamer 30 removes the foams that may be produced in the culture medium during the culturing of the microalgae, thereby ensuring efficient material transport through the membranes (the separation membrane and the hollow fiber membrane) and allowing fast harvesting of the microalgae and supply of nutrients.

For example, the defoamer 30 may be configured as shown in FIG. 1 or FIG. 2. That is, as shown in FIG. 1, it may be provided in plural number along the culture medium flow line such that, after foams are removed from the culture medium discharged from the reactor main body 10 (the culture medium containing relatively large amount of oxygen), foams may be removed again from the culture medium that has passed through the hollow fiber membrane module 20 (the culture medium saturated with carbon dioxide). Alternatively, it may be provided in singular number along the culture medium flow line such that foams may be removed from the culture medium discharged from the reactor main body 10, as shown in FIG. 2.

When the defoamer 30 is provided in singular number as in FIG. 2 such that the culture medium that has passed through the hollow fiber membrane module 20 is circulated directly to the reactor main body 10, the flow rate may be relatively slower as compared to FIG. 1 to prevent membrane fouling. However, there is an advantage in that carbon dioxide can be directly (without passing through the defoamer) supplied to the microalgae.

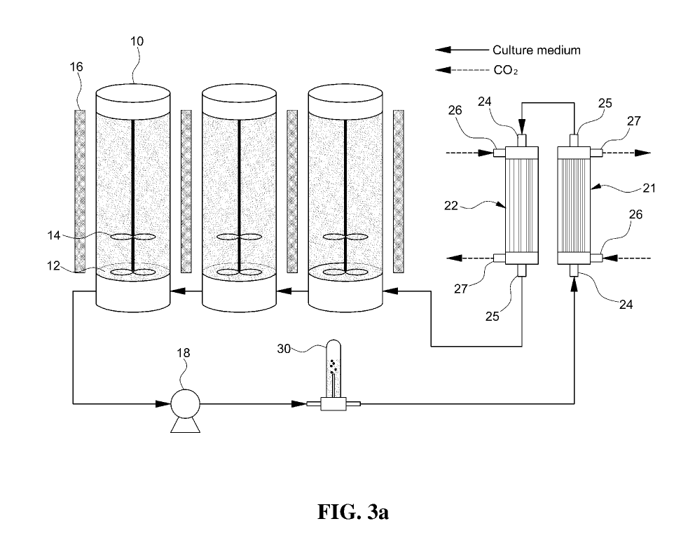

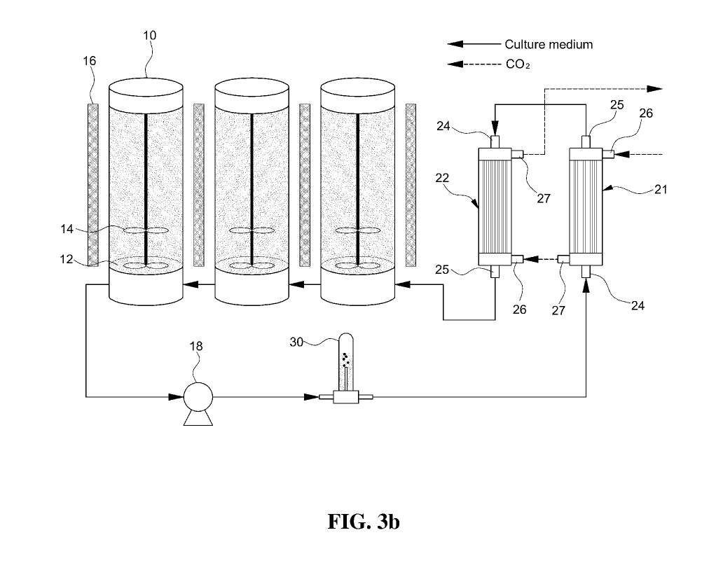

Further, the high-speed photobioreactor according to the illustrative embodiment of the present invention may be configured, as shown in FIG. 3a and FIG. 3b, by modularizing the hollow fiber membrane module 21, 22 and/or the reactor main body 10. When the reactor main body 10 is provided in a plurality, the reactor main bodies 10 may be arranged serially and connected by a culture medium flow line so that the circulating culture medium may sequentially pass through the reactor main bodies 10.

When the reactor main bodies are provided in a plurality, the culture medium may be used in a larger amount than when a single reactor main body is utilized. Thus, the hollow fiber membrane module 21, 22 may be provided serially in a plurality in order to increase carbon dioxide saturation time (or contact time with carbon dioxide and the culture medium). That is to say, as shown in FIG. 3a, the culture medium discharged from the reactor main body 10 is flown into the hollow fiber membrane 23 through the culture medium inlet 24 of the first hollow fiber membrane module 21, and then discharged through the culture medium outlet 25 of the first hollow fiber membrane module 21 after supply of carbon dioxide and removal of oxygen.

Subsequently, the culture medium discharged through the culture medium outlet 25 of the first hollow fiber membrane module 21 is flown again through the culture medium inlet 24 of the second hollow fiber membrane module 22, and then discharged through the culture medium outlet 25 of the second hollow fiber membrane module 22 after supply of carbon dioxide and removal of oxygen. Through this process, the culture medium is saturated with carbon dioxide and then circulated again to the reactor main body 10.

Alternatively, as shown in FIG. 3b, the system may be configured so that the mixture gas discharged from the gas outlet 27 of the first hollow fiber membrane module 21 is flown in to the gas inlet 26 of the second hollow fiber membrane module 22, in order to increase the contact time of carbon dioxide with the culture medium. In this case, the mixture gas discharged after transfer of carbon dioxide to the culture medium in the first hollow fiber membrane module 21 may be reused. Through this process, the saturation degree of carbon dioxide in the culture medium and the removal (fixation) efficiency carbon dioxide in the mixture gas may be increased.

The reuse of the carbon dioxide-containing gas and the carbon dioxide fixation are possible without using an additional collector. That is to say, by further providing the second hollow fiber membrane module 22 between the first hollow fiber membrane module 21 and the reactor main body 10 and then connecting the gas outlet 27 of the first hollow fiber membrane module 21 to the gas inlet 26 of the second hollow fiber membrane module 22, the contact time of carbon dioxide with the culture medium can be increased. As such, by providing the hollow fiber membrane modules 21, 22 serially in a plurality, the contact time of the culture medium with the carbon dioxide gas can be increased and the culture medium can be saturated with carbon dioxide.

After the culture medium saturated with carbon dioxide is supplied to the reactor main body 10, material transport is carried out through the separation membrane 12 due to diffusion by a concentration gradient. At this time, since not only the carbon dioxide but also the oxygen produced by the photosynthesis is diffused, the growth of the microalgae in the culture medium (the microalgae-mixed culture medium) above the separation membrane 12 is improved.

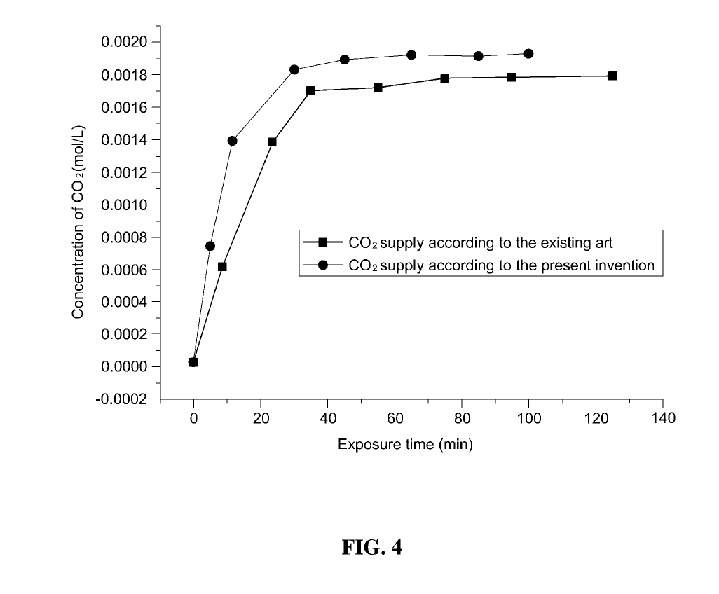

FIG. 4 shows a result, after supplying carbon dioxide at a constant flow rate to the culture medium using the hollow fiber membrane module according to the illustrative embodiment of the present invention or using then existing bubbling reactor, comparing the concentration of carbon dioxide dissolved in each culture medium. The results are shown as a graph with the carbon dioxide concentration in the culture medium shown in the ordinate and the time during which the culture medium is exposed to carbon dioxide (i.e., the time during which the carbon dioxide-containing gas is supplied from the hollow fiber membrane module to the culture medium and dissolved) is shown in the abscissa.

As seen from FIG. 4, when carbon dioxide was supplied to the culture medium through the hollow fiber membrane, carbon dioxide could be dissolved and saturated in the culture medium faster.

As described, since the high-speed photobioreactor according to the present invention uses the hollow fiber membrane having an increased membrane surface area, the saturation rate of carbon dioxide in the circulating culture medium can be increased, and the separation membrane may be installed in the reactor main body to supply carbon dioxide to and remove oxygen from the microalgae-mixed culture medium by the temperature gradient. Furthermore, since the hollow fiber membrane module and the reactor main body can be scaled up by modularization, the growth rate of the microalgae and the carbon dioxide fixation can be maximized.

The present invention has been described in detail with reference to specific embodiments thereof. However, it will be appreciated by those skilled in the art that various changes and modifications may be made in these embodiments without departing from the principles and spirit of the invention, the scope of which is defined in the appended claims and their equivalents.

What is claimed is:

1. A high-speed photobioreactor for culturing microalgae using hollow fiber membranes comprising:
a reactor main body configured to culture microalgae;
a first hollow fiber membrane module configured to supply carbon dioxide into a culture medium in the reactor main body;
a culture medium circulation pump configured to circulate the culture medium; and
a defoamer configured to remove foams produced in the culture medium.

2. The high-speed photobioreactor according to claim 1, wherein the reactor main body is equipped with a separation membrane configured to separate a microalgae-mixed culture medium mixed with microalgae and a circulating culture medium including carbon dioxide supplied from the first hollow fiber membrane module and transferring the carbon dioxide included in the circulating culture medium to the microalgae-mixed culture medium by a concentration gradient.

3. The high-speed photobioreactor according to claim 1, wherein a light source provided outside the reactor main body illuminates light having a wavelength that activates photosynthesis into the reactor main body.

4. The high-speed photobioreactor according to claim 1, wherein one or more stirrer is provided in the reactor main body to ensure flowability of the microalgae.

5. The high-speed photobioreactor according to claim 2, wherein the separation membrane has pores of a size of 0.4 nm or smaller to block the movement of the microalgae through the separation membrane.

6. The high-speed photobioreactor according to claim 1, wherein a hollow fiber membrane of the first hollow fiber membrane module is a hydrophobic membrane having pores of a size 0.1 nm or smaller.

7. The high-speed photobioreactor according to claim 1, wherein a hollow fiber membrane of the first hollow fiber membrane module is a membrane with a porosity of 10-40%.

8. The high-speed photobioreactor according to claim 1, wherein a second hollow fiber membrane module is provided between the first hollow fiber membrane module and the reactor main body, and a gas inlet of the second hollow fiber membrane module is connected to a gas outlet of the first hollow fiber membrane module to increase contact time of carbon dioxide with the culture medium.

9. A photobioreactor comprising:
a reactor configured to culture microalgae;
a first membrane module configured to supply carbon dioxide into a culture medium in the reactor;
a pump configured to circulate the culture medium; and
a defoamer configured to remove foams produced in the culture medium.

10. The photobioreactor of claim 9, wherein the reactor is configured to culture the microalgae in a main body of the reactor.

11. The photobioreactor of claim 9, wherein the membrane module is a first hollow fiber membrane module.

12. The photobioreactor according to claim 11, wherein a second hollow fiber membrane module is provided between the first hollow fiber membrane module and the reactor main body, and a gas inlet of the second hollow fiber membrane module is connected to a gas outlet of the first hollow fiber membrane module to increase contact time of carbon dioxide with the culture medium.

13. The a culture medium circulation photobioreactor of claim 9, wherein the pump is a culture medium circulation pump.

14. The photobioreactor according to claim 9, wherein a hollow fiber membrane of the membrane module has a porosity of 10-40%.

15. The photobioreactor according to claim 9, wherein the reactor is equipped with a separation membrane configured to separate a microalgae-mixed culture medium mixed with microalgae and a circulating culture medium including carbon dioxide supplied from the first membrane module and transferring the carbon dioxide included in the circulating culture medium to the microalgae-mixed culture medium by a concentration gradient.
